(19) United States
(12) Patent Application Publication
(10) Pub. No.: US 2025/0018116 A1
(43) Pub. Date: Jan. 16, 2025

(54) LIGHT-BASED VISUAL CUEING OF MEDICATION DELIVERY INSTRUCTIONS

(71) Applicant: Janssen Research & Development, LLC, Raritan, NJ (US)

(72) Inventor: Jason TANSKY, Raritan, NJ (US)

(21) Appl. No.: 18/706,585

(22) PCT Filed: Oct. 17, 2022
(86) PCT No.: PCT/IB2022/059960

§ 371 Date: May 1, 2024

Related U.S. Application Data
(60) Provisional application No. 63/275,837, filed on Nov. 4, 2021.

(51) Int. Cl. A61M 5/20

(52) U.S. Cl. CPC A61M 5/20; A61M 2005/208; A61M 2205/3561; A61M 2205/3584; A61M 2205/3592; A61M 2205/502; A61M 2205/581; A61M 2205/584

(57) ABSTRACT

A wireless receiver may be configured to receive a plurality of wireless communications from a computing device. A first light source may be activated based on a first wireless communication of the plurality of wireless communications. Activating of the first light source may be a first visual cue to perform a first step of a plurality of steps of instructions for use of a medication delivery device, and the first light source may be activated in correspondence with the first step being displayed by the computing device. A second light source may be activated based on a second wireless communication of the plurality of wireless communications. Activating of the second light source may be a second visual cue to perform a second step of the plurality of steps, and the second light source may be activated in correspondence with the second step being displayed by the computing device.

### TECHNICAL FIELD

The present disclosure relates generally to providing light-based visual cues, for example to assist a patient in proper use of a medication delivery device, such as a medication injection device. Light sources, such as an organic light-emitting diode (OLED), may be activated to cue the user to perform various medication delivery instructions, such as in a corresponding sequence.

### BACKGROUND

A medication delivery device may be used to store and/or deliver a medication. In some examples, a medication delivery device may be a medication injection device, such as a prefilled syringe, an autoinjector, or wearable infusion pump. In some other examples, a medication delivery device may be a medication storage container, such as a bottle, a blister pack or other medication packaging.

Patients may often not perform an injection properly, such as by failing to remove a cap of the medication delivery device, not sufficiently pressing down on a medication delivery device to inject a complete dosage, failing to lift the medication delivery device up after injection, and the like. Additionally, patients may sometimes neglect to perform other medication delivery steps, such as shaking a medication before use.

Medication delivery instructions may sometimes be printed on a product label or instruction sheet. However, users may often neglect to read the printed instructions or may not completely comprehend the instructions. Moreover, it may be unclear from printed instructions exactly which step of a process the user is currently on, thereby potentially resulting in missed, or otherwise improperly performed, steps and instructions.

### SUMMARY

An example light-based medication delivery cueing system is described. The system may include a wireless receiver configured to receive a plurality of wireless communications from a computing device via a wireless communication protocol. The system may further include a first light source that is activated based on a first wireless communication of the plurality of wireless communications. Activating of the first light source may be a first visual cue to perform a first step of a plurality of steps of instructions for use of a medication delivery device. The first light source may be activated in correspondence with the first step being displayed by the computing device. The system may further include a second light source that is activated based on a second wireless communication of the plurality of wireless communications. Activating of the second light source may be a second visual cue to perform a second step of the plurality of steps associated with the instructions for use. The second light source may be activated in correspondence with the second step being displayed by the computing device. The second wireless communication may be transmitted based on user input to the computing device.

An example light-based medication delivery cueing method is also described. The method may include receiving, by a wireless receiver, from a computing device, via a wireless communication protocol, a first wireless communication. The method may further include activating, based on the first wireless communication, a first light source. Activating of the first light source may be a first visual cue to perform a first step of a plurality of steps of instructions for use of a medication delivery device. The first light source may be activated in correspondence with the first step being displayed by the computing device. The method may further include receiving, by the wireless receiver, from the computing device, via the wireless communication protocol, a second wireless communication. The second wireless communication may be transmitted based on user input to the computing device. The method may further include activating, based on the second wireless communication, a second light source. Activating of the second light source may be a second visual cue to perform a second step of the plurality of steps associated with the instructions for use. The second light source may be activated in correspondence with the second step being displayed by the computing device.

### BRIEF DESCRIPTION OF THE DRAWINGS

For a more complete understanding of the principles disclosed herein, and the advantages thereof, reference is now made to the following descriptions taken in conjunction with the accompanying drawings, in which:

FIG. 1A is a front elevation view of a first example medication delivery device in a pre-use position.

FIG. 1B is a front elevation view of the first example medication delivery device with the cap removed so as expose the needle guard.

FIG. 1C is a front elevation view of the first example medication delivery device with the needle guard moved from its position in FIG. 1B.

FIG. 1D is a front elevation view of the first example medication delivery device with the upper housing moving toward the dispensed position.

FIG. 1E is a front elevation view of the first example medication delivery device with the upper housing in the dispensed position.

FIG. 1F is a front elevation view of the first example medication delivery device with the needle guard in the final position.

FIG. 2 is a view of a second example medication delivery device that is an example pre-filled syringe.

FIG. 3 is a view of a third example medication delivery device that is an example autoinjector.

FIG. 4 is a view of a fourth example medication delivery device that is an example wearable medication delivery device.

FIG. 5 is a view of a first example computing device page and light sources integrated with the first example medication delivery device.

FIG. 6 is a view of a second example computing device page and example corresponding light source activation.

FIG. 7 is a view of a third example computing device page and example corresponding light source activation.

FIG. 8 is a view of a fourth example computing device page and example corresponding light source activation.

FIG. 9 is a view of a fifth example computing device page and example corresponding light source activation.

FIG. 10 is a view of a sixth example computing device page and example corresponding light source activation.

FIG. 11 is a diagram of an example light-based medication delivery cueing system.

FIG. 12 is a flowchart of an example light-based medication delivery cueing process.

### DETAILED DESCRIPTION

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims. Further, the term “at least one” stated structure as used herein can refer to either or both of a single one of the stated structure and a plurality of the stated structure. Additionally, reference herein to a singular “a,” “an,” or “the” applies with equal force and effect to a plurality unless otherwise indicated. Similarly, reference to a plurality herein applies with equal force and effect to the singular “a,” “an,” or “the.”

References in the specification to “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

### Example Medication Delivery Devices

Some example medication delivery devices will now be described in association with which the light-based visual cueing techniques described may be implemented. The term medication delivery device, as used herein, refers to a device that is used to store and/or deliver a medication. Medication delivery devices may include, for example, medication injection devices and medication storage containers. In some examples, the light-based visual cueing techniques described herein may be implemented in association with medication injection devices such as autoinjectors, prefilled syringes, wearable medication delivery devices (e.g., patch pumps, on body delivery systems (OBDS's), etc.), and others. It is noted, however, that the techniques described herein are not limited to use with any particular types of medication delivery devices, and a wide variety of types of medication delivery devices may be employed.

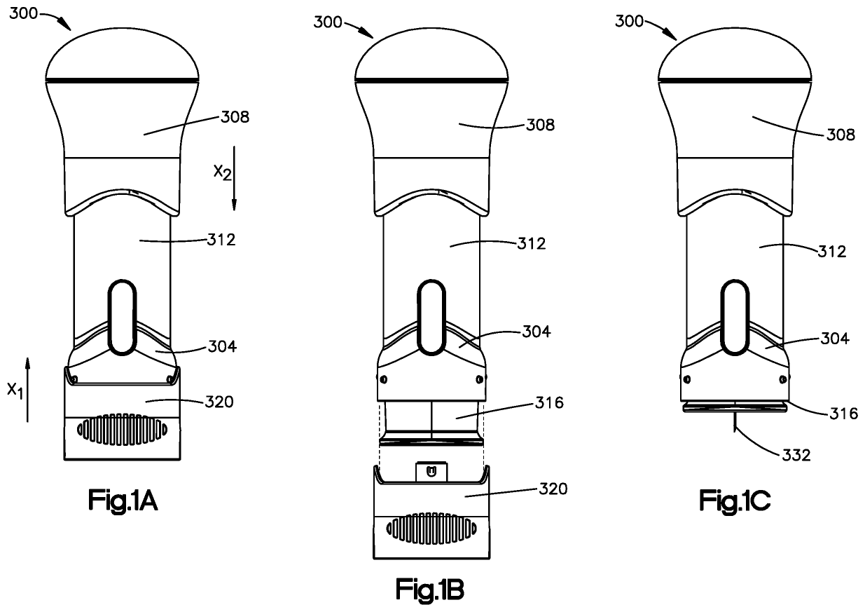

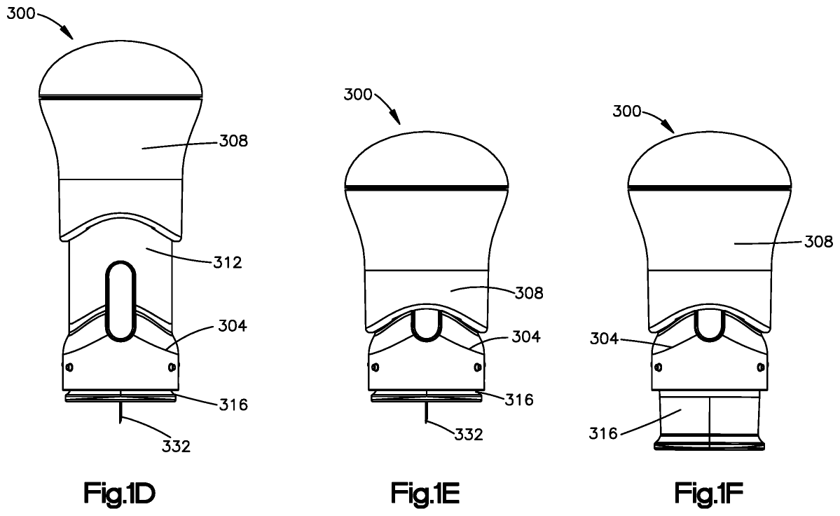

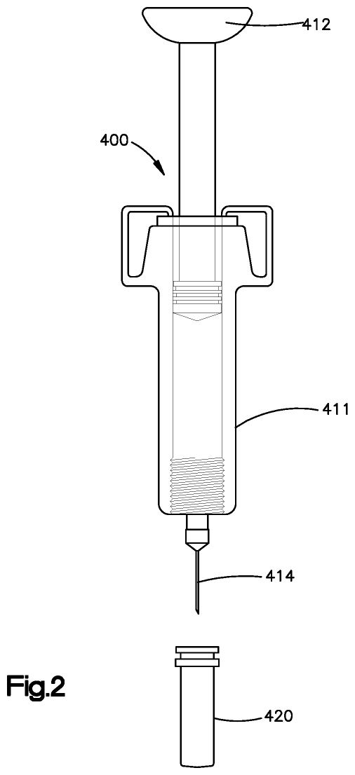

Referring now to FIGS. 1A-1F, a first example medication delivery device is described in association with which the light-based visual cueing techniques described may be implemented. As will be described, the device 300 may be employed to inject a medication, such as by pressing down on an upper housing 308 and moving the upper housing from a pre-use position to a dispensed position. In operation and in reference to FIGS. 1A-1F, the device 300 can be configured to deliver a medication. Prior to use, the upper housing 308 can be locked in the pre-use position, and the cap 320 can be coupled to the lower housing 304 so as to shield the needle guard 316 and the needle 332. When the device 300 is ready to be used, the cap 320 can be removed from the lower housing 304 as shown in FIG. 1B.

As shown in FIG. 1C, the device 300 can be positioned against a skin surface and a manual force can be applied to the upper housing 308 along an insertion direction, which is direction X₂ in FIG. 1A, such that, as the needle guard 316 is pressed against the skin surface, the needle guard 316 moves and the needle 332 is inserted into the tissue. As the needle guard 316 moves, the upper housing 308 may be unlocked from the pre-use position. As shown in FIGS. 1D and 1E, the upper housing 308 can then be moved along the insertion direction and over the middle housing 312. The upper housing 308 of device 300 is supported relative to the lower housing 304 and is configured to receive a manual force and move with respect to the lower housing 304 in the insertion direction from the pre-use position to the dispensed position in response to the manual force. The device 300 may include an internal syringe that is supported by the lower housing 304 and a plunger rod that is carried by the upper housing 308 and movable with the upper housing 308 so as to advance relative to the syringe when the upper housing 308 is moved along the insertion direction. The syringe may retain a medication and carry the needle 332 that is configured to be inserted into tissue. Advancement of the plunger rod relative to the syringe may cause the syringe to deliver the medication out the needle 332 and into the tissue. When the upper housing 308 reaches the dispensed position, the upper housing 308 may be locked in the dispensed position, for example via internal locking latches and latch members, so as to prevent re-use of the device 300. As the upper housing 308 is locked in dispensed position, such as by locking latches snapping over the latch members, an audible click may be produced that signifies to the user that the upper housing 308 has reached the dispensed position and is locked in the dispensed position. The upper housing 308 can be permanently locked in the dispensed position such that the device 300 is not reusable. It should be appreciated, however, that the upper housing 308 can be temporarily locked such that the device 300 can be sterilized and reused.

As shown in FIG. 1F, when the device 300 is removed from the skin surface along a direction X₁ opposite the insertion direction the needle guard 316 moves along the insertion direction to the final position. When in the final position, the needle guard 316 can be permanently locked in the final position so that the device 300 is not reusable. It should be appreciated, however, that the needle guard 316 can be temporarily locked such that the device 300 can be sterilized and reused.

Thus, device 300, which is described above with reference to FIGS. 1A-1F, is one example of a medication delivery device in association with which the light-based visual cueing techniques described herein may be implemented. However, the light-based visual cueing techniques described herein may also be implemented in association with other medication delivery devices, such as autoinjectors, prefilled syringes, wearable medication delivery devices (e.g., patch pumps, OBDS's), and others.

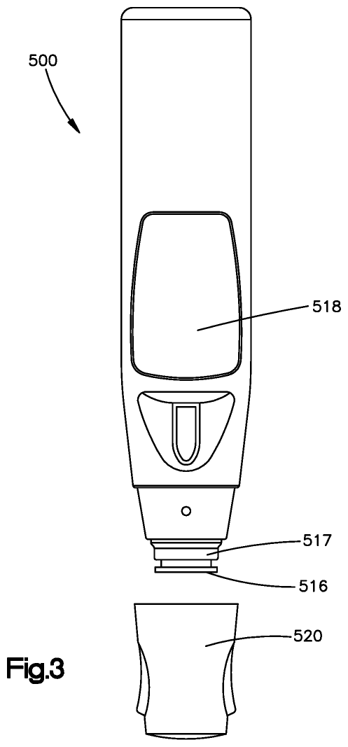

Referring now to FIG. 2, device 400 is an example of a prefilled syringe in association with which the light-based visual cueing techniques described herein may be implemented. As shown in FIG. 2, device 400 has a cap 420, which may be removed by the user when the user is ready to inject a medication using the device 400. After removing the cap 420, the device 400 may be held, and the needle 414 may be inserted. The user may then place his or her thumb on the plunger 412 and press the plunger 412 all the way down until the plunger 412 stops. The user may then release pressure from the plunger 412. The needle 414 may then retract into the body 411. Thus, in device 400, the user presses down on the plunger 412, for example as opposed to upper housing 308 of device 300 of FIG. 3.

Referring now to FIG. 3, device 500 is an example of an autoinjector in association with which the light-based visual cueing techniques described herein may be implemented. As shown in FIG. 2, device 500 has a cap 520, which may be removed by the user when the user is ready to inject a medication using the device 500. After removing the cap 520, the user may hold device 500 and position the device 500 against the skin with the safety sleeve 516 flat against the skin. The user may then push firmly against the skin, which may cause the safety sleeve 516 to slide into the cover 517. The user may then press button 518, which may cause a medication to be injected into the user via a hidden needle, such as may be located adjacent to safety sleeve 516 and cover 517. The device 500 may produce an audible sound, such as a first click sound, as the button 518 is pressed and the injection of the medication is started. The user may continue to press button 518 until the medication is fully dispensed. Similar to device 300 of FIG. 3, device 500 may also produce an audible sound, such as a click sound, once the medication has been fully dispensed, and this second click sound may be an indication to the user that the medication has been fully dispensed. Upon hearing the second click sound, the user may lift the device 500 from the skin. Thus, one way in which device 500 differs from device 300 is that device 500 allows the injection to be triggered by pressing a button 518.

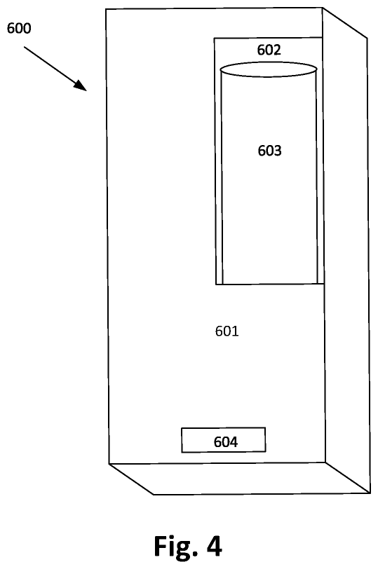

Referring now to FIG. 4, device 600 is an example of a wearable medication delivery device in association with which the light-based visual cueing techniques described herein may be implemented. Device 600 may be, or may be included in, an on body delivery system (OBDS). FIG. 4 shows a front surface 601 of device 600. In some examples, device 600 may be attachable to a user's skin. For example, in some cases, a rear surface of device 600, which may be opposite front surface 601 (and which is not shown in FIG. 4), may have an attached adhesive pad that may allow attachment of device 600 to a user's skin. Also, in some examples, device 600 may have an associated strap for assistance in attaching device 600 to a user's skin. As shown in FIG. 4, a medication tube 603 is visible through window 602. In operation, device 600 may cause a medication to be delivered from medication tube 603 to a user via an injection. In the example of FIG. 4, a user may press button 604 to cause the injection process to start. Device 600 may include a needle, such as may protrude from the rear of device 600, through which injection of the medication may be delivered.

Thus, as described above, the light-based visual cueing techniques described herein may be implemented in association with medication delivery devices such as autoinjectors, prefilled syringes, wearable medication delivery devices (e.g., patch pumps, on body delivery systems (OBDS's), etc.), and others. However, it is again noted that the techniques described herein are not limited to use with any particular types of medication delivery devices, and a wide variety of types of medication delivery devices may be employed. As another example, the light-based visual cueing techniques described herein may also be used in combination with other medication delivery devices, such as medication storage containers (e.g., storage bottles, blister packs and other storage packages, etc.).

### Light-Based Visual Cueing of Medication Delivery Instructions

As described herein, a light-based medication delivery cueing system may include a lighting circuit that is integrated with a medication delivery device. The lighting circuit may include light sources. In some examples, in order to not cause substantial changes to the size and/or shape of the medication delivery device, any or all of the light sources described herein may be a thin, flat and/or flexible component, such as an organic light-emitting diode (OLED) and/or OLED display. Also, in some examples, other light sources may be employed, such as a light-emitting diode (LED), an LED display, other thin film type lights, incandescent lights, and/or other light sources. As also described herein, the lighting circuit may include a wireless receiver that receives wireless communications, from a computing device (e.g., smartphone, tablet, laptop, etc.), via a wireless communication protocol, such as Bluetooth, Near-Field Communication (NFC), and the like. The light sources may be activated based communications received from the computing device. The light sources may be activated in order to assist users with the injection process, such as by providing visual cues to perform various steps of the injection process (e.g., remove cap, place device on skin, push handle down, push until click, lift up, etc.). Activating a light source means that the light source is caused to assume one or more active states, such as to start emitting light (e.g., by being powered-on), to start flashing, to change color (e.g., to change to a green color or another designated color associated with an active state), and the like.

Patients may often not perform an injection properly, such as by failing to remove a cap of the medication delivery device, not sufficiently pressing down on a medication delivery device to inject a complete dosage, failing to lift the medication delivery device up after injection, and the like. By using light-based cueing of steps of the injection process, the likelihood is reduced that users may perform the injection process improperly.

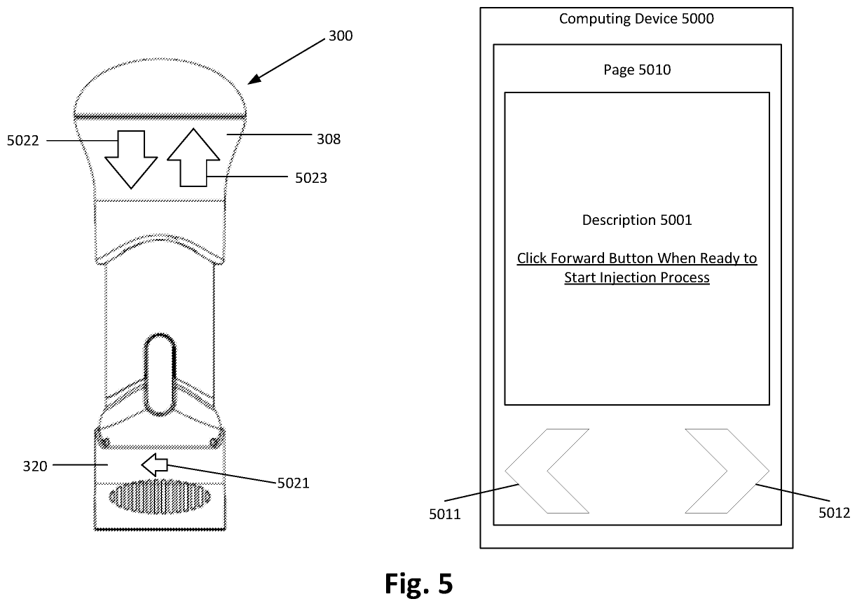

Referring now to FIG. 5, an example is shown in which light source 5021, light source 5022 and light source 5023 (referred to collectively as light sources 5021-5023) are integrated with device 300. In some examples, the light sources 5021-5023 may be included in one or more adhesive labels that are attached to the device 300. In other examples, light sources 5021-5023 may be components of the device 300. In this example, light source 5021 is located on the cap 320. Light sources 5022 and 5023 are located on the upper housing 308. Light source 5021 has the shape of a horizontal arrow, which is a cue to the user to remove the cap 320. Light source 5022 is down arrow, which is a cue to the user to push down on the device 300. Light source 5023 is an up arrow, which is a cue to the user to lift the device 300 up. FIG. 5 depicts light sources 5021-5023 prior to the start of the injection of the medication. Thus, in the example of FIG. 5, none of the light sources 5021-5023 are activated.

FIG. 5 also shows a computing device 5000, such as a smartphone, tablet or laptop. The computing device displays a page 5010. The page 5010 includes a back button 5011 and a forward button 5012. The user may select (e.g., click) the back button 5011 to indicate that the he or she wants to return to a prior step in the injection process. By contrast, the user may select (e.g., click) the forward button 5012 to indicate that he or she is ready to advance to a next step in the injection process. The page 5010 includes a description 5001, which includes text indicating a current step in the injection process for the user to perform. In some examples, the description 5001 may include text and/or images describing the corresponding step in the injection process. The description 5001 indicates that the user should click the forward button 5012 when the user is ready to start the injection process.

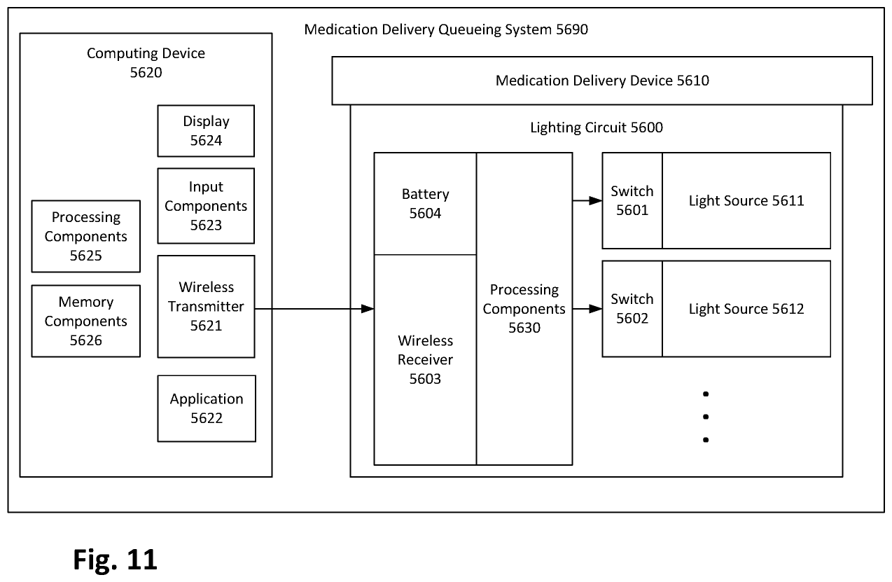

As will be described in detail below with reference to FIG. 11, the computing device 5000 may send, via wireless transmitter 5621, wireless communications to a wireless receiver 5603 of FIG. 11 that may be integrated with a medication delivery device 5610, such as device 300. Thus, it is appreciated that communications sent from computing device 5000 to device 300, which are described with reference to FIGS. 5-10, may be performed via wireless transmitter 5621 and wireless receiver 5603 of FIG. 11. When the user selects forward button 5012 from page 5010, this indicates that the user is ready to start the injection process. In response to this user input (e.g., selection of forward button 5012 from page 5010), the computing device 5000 may send a communication to the device 300 indicating that light source 5021 should be activated.

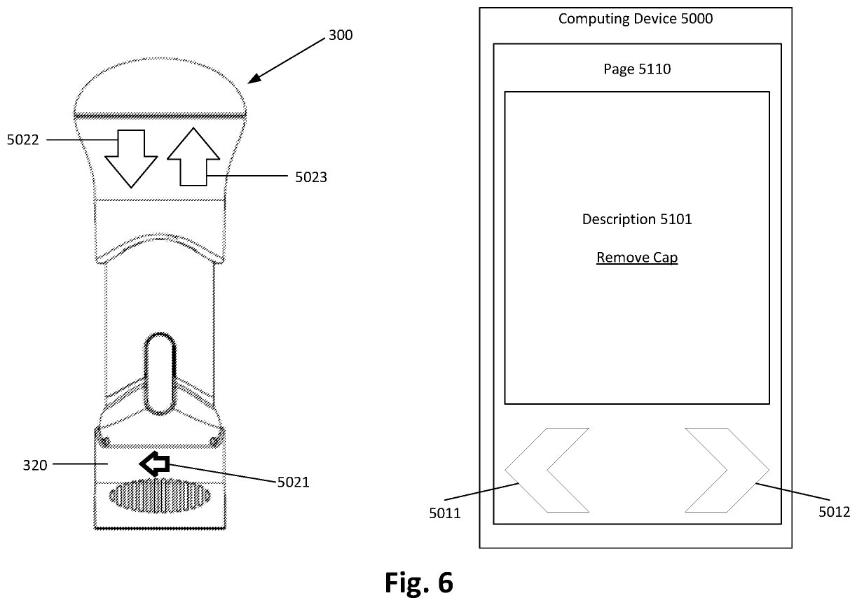

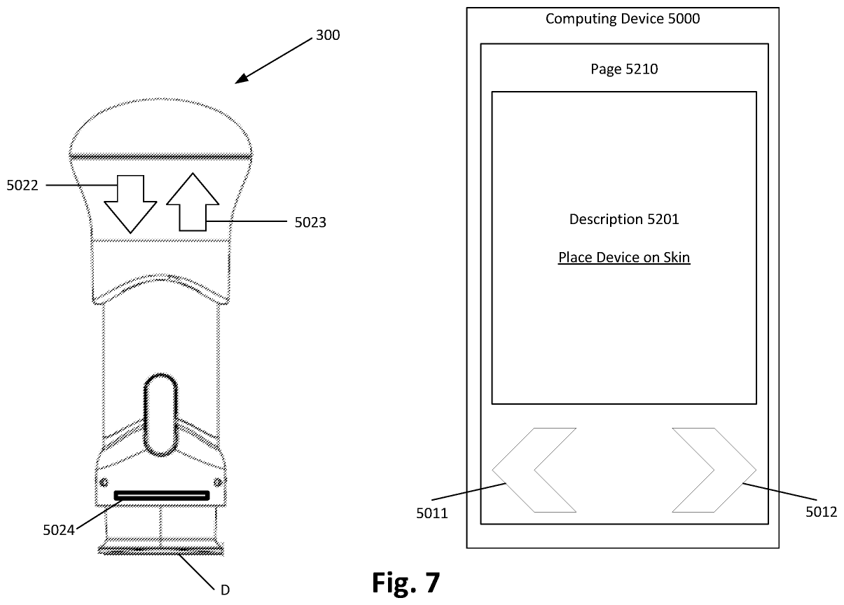

Referring now to FIG. 6, an example is shown of device 300 after the user selects the forward button 5012 from page 5010 of FIG. 5. In this example, the first step of the injection process is for the user to remove the cap 320 of device 300. Accordingly, in FIG. 6, light source 5021 is activated in order to cue the user to remove the cap 320. As described above, light source 5021 has the shape of a horizontal arrow, which is a cue to the user to remove the cap 320. In FIG. 6, light source 5021 is shown with thick/bold outlining to indicate that the light source 5021 is currently activated. By contrast, in FIG. 6, light sources 5022 and 5023 have thin outlining, which indicates that they are not activated. Light source 5021 (as well as other light sources described herein) may be activated by being changed to one or more active states, such as to start emitting light (e.g., by being powered-on), to start flashing, to change color (e.g., to change to a green color or another designated color associated with an active state), and the like

As also shown in FIG. 6, the selection of forward button 5012 from page 5010 of FIG. 5 has caused the computing device 5000 to load a subsequent page (e.g., page 5110). The page 5110 includes a description 5101, which includes text indicating a current step in the injection process for the user to perform. In some examples, the description 5101 may include text and/or images describing the corresponding step in the injection process. The description 5001 indicates that the user should remove the cap 320.

Thus, activation of light source 5021 may cue the user to remove the cap 320. After removing the cap 320, the user may select forward button 5012 from page 5110. When the user selects forward button 5012 from page 5110, this indicates that the user has successfully removed the cap 320 from device 300. In response to this user input (e.g., selection of forward button 5012 from page 5110), the computing device 5000 may send a communication to the device 300 indicating that light source 5021 should be deactivated. Deactivating of a light source means that it the light source is removed from a state that it assumed when it was activated, such as by being caused to stop emitting light (e.g., by being powered-off), to stop flashing, to change color (e.g., to change back to a color it had before it was activated), and the like. Additionally, in response to this user input (e.g., selection of forward button 5012 from page 5110), the computing device 5000 may send a communication to the device 300 indicating that light source 5024 (shown in FIG. 7) should be activated. The communications to deactivate light source 5021 and to activate light source 5024 may be sent via a single combined communication or multiple communications.

Referring now to FIG. 7, an example is shown of device 300 after the user selects the forward button 5012 from page 5110 of FIG. 6. In this example, the next step of the injection process is for the user to place the device 300 on his or her skin. Accordingly, in FIG. 7, light source 5024 is activated in order to cue the user to place the device 300 on his or her skin. It is noted that the light source 5024 is obscured by cap 320 (and therefore not shown) in FIGS. 5 and 6. In some examples, the light source 5024 may alternatively be positioned closer to the distal end D of the device 300. In FIG. 7, light source 5024 is shown with thick/bold outlining to indicate that the light source 5024 is currently activated. By contrast, in FIG. 7, light sources 5022 and 5023 have thin outlining, which indicates that they are not activated.

As also shown in FIG. 7, the selection of forward button 5012 from page 5110 of FIG. 6 has caused the computing device 5000 to load a subsequent page (e.g., page 5210). The page 5210 includes a description 5201, which includes text indicating a current step in the injection process for the user to perform. In some examples, the description 5201 may include text and/or images describing the corresponding step in the injection process. The description 5201 indicates that the user should place the device 300 on his or her skin.

Thus, activation of light source 5024 may cue the user to place the device 300 on his or her skin. After placing the device 300 on his or her skin, the user may select forward button 5012 from page 5210. When the user selects forward button 5012 from page 5210, this indicates that the user has successfully placed the device 300 on his or her skin. In response to this user input (e.g., selection of forward button 5012 from page 5210), the computing device 5000 may send a communication to the device 300 indicating that light source 5024 should be deactivated. Additionally, in response to this user input (e.g., selection of forward button 5012 from page 5210), the computing device 5000 may send a communication to the device 300 indicating that light source 5022 should be activated. The communications to deactivate light source 5024 and to activate light source 5022 may be sent via a single combined communication or multiple communications.

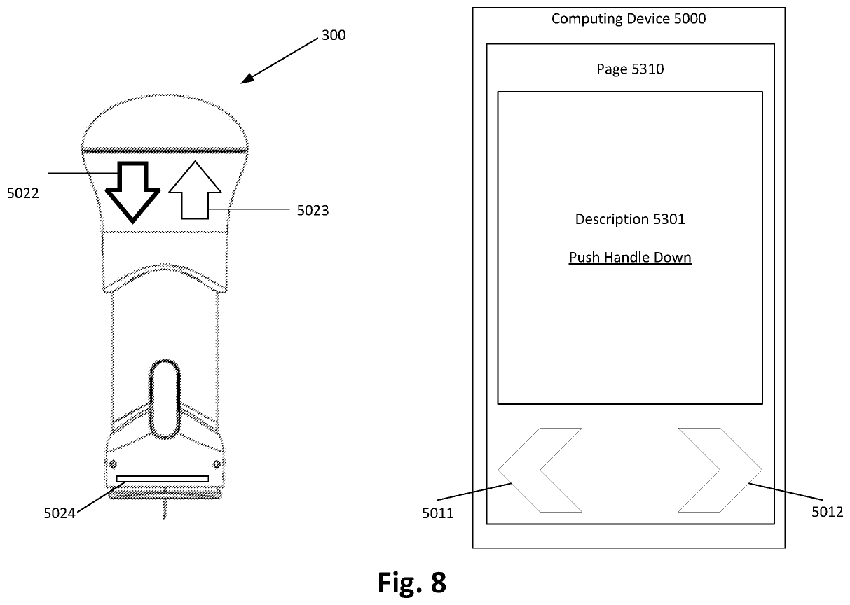

Referring now to FIG. 8, an example is shown of device 300 after the user selects the forward button 5012 from page 5210 of FIG. 7. In this example, the next step of the injection process is for the user to push the handle (e.g., the handle shape formed by upper housing 308) down. Accordingly, in FIG. 8, light source 5022 is activated in order to cue the user to push the handle down. As described above, light source 5022 has the shape of a down arrow, which is a cue to the user to push the handle down. In FIG. 8, light source 5022 is shown with thick/bold outlining to indicate that the light source 5022 is currently activated. By contrast, in FIG. 8, light sources 5023 and 5024 have thin outlining, which indicates that they are not activated.

As also shown in FIG. 8, the selection of forward button 5012 from page 5210 of FIG. 7 has caused the computing device 5000 to load a subsequent page (e.g., page 5310). The page 5310 includes a description 5301, which includes text indicating a current step in the injection process for the user to perform. In some examples, the description 5301 may include text and/or images describing the corresponding step in the injection process. The description 5301 indicates that the user should push the handle down.

Thus, activation of light source 5022 may cue the user to push the handle down. After pushing down on the handle, the user may select forward button 5012 from page 5310. When the user selects forward button 5012 from page 5310, this indicates that the user has pushed the handle down.

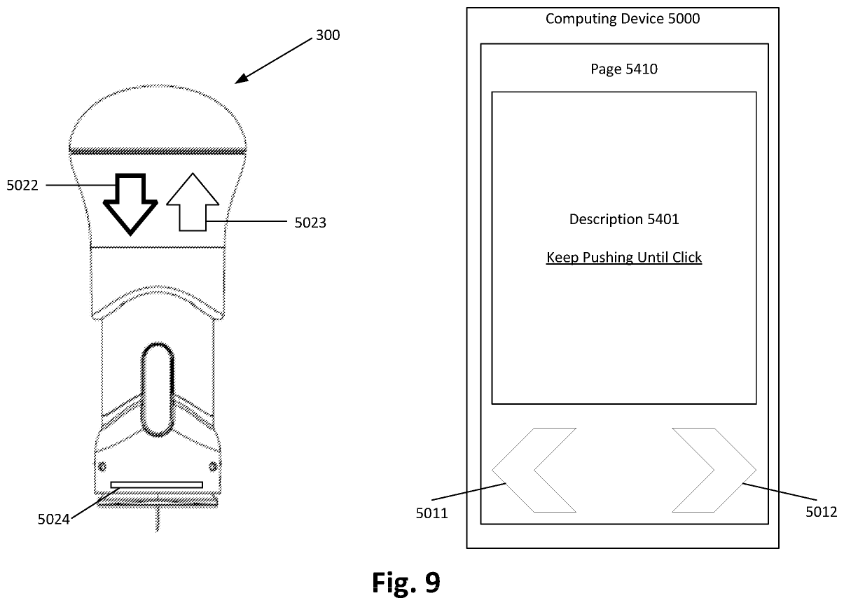

Referring now to FIG. 9, an example is shown of device 300 after the user selects the forward button 5012 from page 5310 of FIG. 8. In this example, the next step of the injection process is for the user to keep pushing on the handle until a click is heard. For example, as described above, the device 300 may make a click sound when injection of a medication is completed. Specifically, as described above, such as in relation to FIGS. 1A-1F, as the upper housing 308 is locked in the dispensed position, such as by locking latches snapping over the latch members, an audible click may be produced that signifies to the user that the upper housing 308 has reached the dispensed position and is locked in the dispensed position. Accordingly, in FIG. 9, light source 5022 remains activated in order to cue the user to keep pushing on the handle until a click is heard. As described above, light source 5022 has the shape of a down arrow, which is a cue to the user to keep pushing on the handle until a click is heard. In FIG. 9, light source 5022 is shown with thick/bold outlining to indicate that the light source 5022 is currently activated. By contrast, in FIG. 9, light sources 5023 and 5024 have thin outlining, which indicates that they are not activated.

As also shown in FIG. 9, the selection of forward button 5012 from page 5310 of FIG. 8 has caused the computing device 5000 to load a subsequent page (e.g., page 5410). The page 5410 includes a description 5401, which includes text indicating a current step in the injection process for the user to perform. In some examples, the description 5401 may include text and/or images describing the corresponding step in the injection process. The description 5401 indicates that the user should keep pushing until a click is heard.

After the user hears the click, the user may select forward button 5012 from page 5410. When the user selects forward button 5012 from page 5410, this indicates that the user has heard the click and the dosage of the medication has been fully injected. In response to this user input (e.g., selection of forward button 5012 from page 5410), the computing device 5000 may send a communication to the device 300 indicating that light source 5022 should be deactivated. Additionally, in response to this user input (e.g., selection of forward button 5012 from page 5410), the computing device 5000 may send a communication to the device 300 indicating that light source 5023 should be activated. The communications to deactivate light source 5022 and to activate light source 5023 may be sent via a single combined communication or multiple communications.

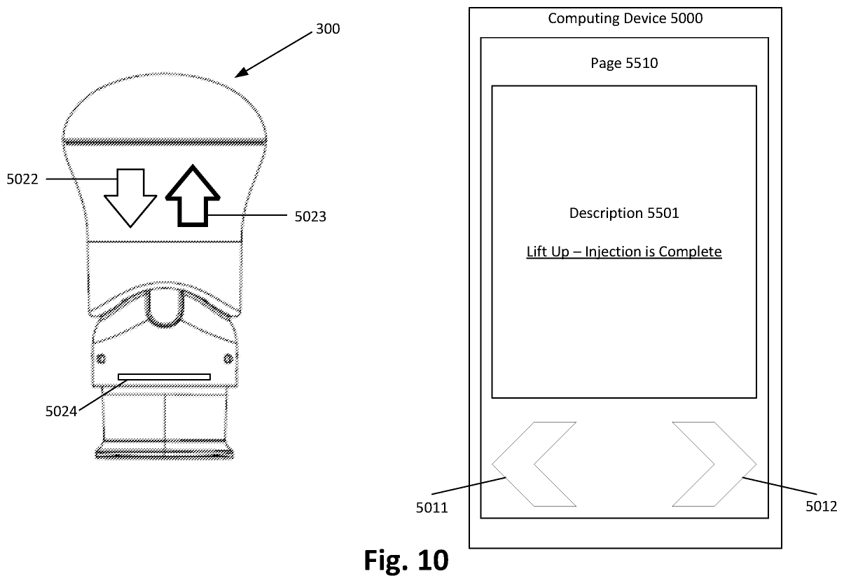

Referring now to FIG. 10, an example is shown of device 300 after the user selects the forward button 5012 from page 5410 of FIG. 9. In this example, the next step of the injection process is for the user to lift the device 300 up. Accordingly, in FIG. 9, light source 5023 is activated in order to cue the user to lift the device 300 up. As described above, light source 5023 has the shape of an up arrow, which is a cue to the user to lift the device 300 up. In FIG. 10, light source 5023 is shown with thick/bold outlining to indicate that the light source 5023 is currently activated. By contrast, in FIG. 10, light sources 5022 and 5024 have thin outlining, which indicates that they are not activated.

As also shown in FIG. 10, the selection of forward button 5012 from page 5410 of FIG. 9 has caused the computing device 5000 to load a subsequent page (e.g., page 5510). The page 5510 includes a description 5501, which includes text indicating a current step in the injection process for the user to perform. In some examples, the description 5501 may include text and/or images describing the corresponding step in the injection process. The description 5501 indicates that the user should lift the device 300 up. The description 5501 also indicates that the injection is complete.

Thus, activation of light source 5023 may cue the user to lift the device 300 up. The activation of light source 5023 is also a visual indicator on the device 300 that the injection is complete. After lifting the device 300 up, the user may select forward button 5012 from page 5510. When the user selects forward button 5012 from page 5510, this indicates that the user has lifted the device 300 up. In response to this user input (e.g., selection of forward button 5012 from page 5510), the computing device 5000 may send a communication to the device 300 indicating that light source 5023 should be deactivated.

Referring now to FIG. 11, an example of a light-based medication delivery cueing system 5690 will now be described in detail. As shown, light-based medication delivery cueing system 5690 includes lighting circuit 5600, medication delivery device 5610 and computing device 5620. In one specific example, medication delivery device 5610 may be device 300 of FIGS. 1A-1F.

The lighting circuit 5600 may be integrated with the medication delivery device 5610. The term integrated with, as used herein, means that the lighting circuit 5600 is included within the medication delivery device 5610 or is otherwise directly, or indirectly (e.g., via one or more connecting or attaching components), physically connected, or attached, to (e.g., via an adhesive, etc.) the medication delivery device 5610, before and/or at the time that the medication delivery device is used by a patient (and/or by another user on the patient's behalf). Thus, battery 5604, wireless receiver 5603, processing components 5630, switch 5601, light source 5611, switch 5602, and light source 5612 may be integrated with the medication delivery device 5610. In some examples, the lighting circuit 5600 may be included in one or more thin and flexible adhesive labels that are attachable to, and/or attached to, the medication delivery device 5610. Thus, in some examples, any, or all, of battery 5604, wireless receiver 5603, processing components 5630, switch 5601, light source 5611, switch 5602, and light source 5612 may be included in one or more thin and flexible adhesive label that are attachable to, and/or attached to, the medication delivery device 5610. Additionally, in some examples, the lighting circuit 5600 may be included within the medication delivery device 5610. For example, the lighting circuit 5600 may be embedded into one or more other components of the medication delivery device 5610, such as by being molded into plastic and/or other materials of which those components may be comprised. Thus, in some examples, any, or all, of battery 5604, wireless receiver 5603, processing components 5630, switch 5601, light source 5611, switch 5602, and light source 5612 may be included within the medication delivery device 5610.

In some examples, the computing device 5620 may be a smartphone, a tablet, or a laptop. In this example, the computing device 5620 includes a display 5624, such as a touchscreen, monitor or other display screen. The display 5624 may be used to display pages 5010, 5110, 5210, 5310, 5410 and 5510 of FIGS. 5-10. These pages may be displayed via a graphical user interface (GUI). The computing device 5620 also includes input component 5623, via which user input may be received. In some examples, the display 5624 may be also input component 5623, such as when the display 5624 is a touchscreen. In other examples, input component 5623 may be a different component, such as a mouse or keyboard. The computing device 5620 also includes application 5622, which may generate pages 5010, 5110, 5210, 5310, 5410 and 5510 of FIGS. 5-10. The application 5622 may also receive user inputs, such as selections of forward button 5012. As described in detail below, in response to user inputs, the application 5622 may cause wireless communications to be transmitted to wireless receiver 5603, for example including instructions to activate light sources 5611 and 5612. Application 5622 may be a web browser or another application, such as an application that specifically allows for interaction with, and control of, lighting circuit 5600. Wireless transmitter 5621 transmits wireless communications via a wireless communication protocol, such as a short-range wireless communication protocol (e.g., Bluetooth, Near-Field Communication (NFC), and the like).

Computing device 5620 further includes processing components 5625 and memory components 5626. A computing device, as that term is used herein, refers to a device including at least processing components 5625 and memory components 5626 that is programmable to perform computing operations. Processing components 5625 may be general purpose computer processors. The memory components 5626 can be volatile (such as some types of RAM), non-volatile (such as ROM, flash memory, etc.), or a combination thereof. The memory components 5626 can include additional storage (e.g., removable storage and/or non-removable storage) including, but not limited to, tape, flash memory, smart cards, CD-ROM, digital versatile disks (DVD) or other optical storage, magnetic cassettes, magnetic tape, magnetic disk storage or other magnetic storage devices, universal serial bus (USB) compatible memory, or any other medium which can be used to store information and which can be accessed by the computing device 5620. The memory components 5626 can have stored therein instructions that, upon execution by the processing components 5625, cause the computing device 5620 to perform operations, such as the operations performed by computing device 5620 as described herein.

Lighting circuit 5600 includes a battery 5604, wireless receiver 5603, processing components 5630, switches 5601 and 5602 and light sources 5611 and 5612. Battery 5604 may power wireless receiver 5603, processing components 5630, and, when activated, light sources 5611 and 5612. Processing components 5630 may be components that are programmed, or otherwise instructed, to perform the operations that they execute as described herein, and that may be included in one or more integrated circuit (IC) chips and/or other IC components. Wireless receiver 5603 is configured to receive a plurality of wireless communications from computing device 5620 (e.g., from wireless transmitter 5621) via a wireless communication protocol. In some examples, light source 5611 may be any of light sources 5021, 5022, 5023 and 5024 described above. Additionally, in some examples, light source 5612 may be any of light sources 5021, 5022, 5023 and 5024 described above. Moreover, in some examples, lighting circuit 5600 may optionally include any number of additional light sources and any number of additional switches (as indicated by the ellipsis shown in FIG. 11).

Specifically, a user may provide first user input to application 5622 of computing device 5620. In one specific example, the first user input may indicate that the user is ready to start the injection process. For example, this may include a selection of forward button 5012 of page 5010 of FIG. 5. Upon receipt of the first user input, the application 5622 may cause the wireless transmitter 5621 to transmit a first wireless communication of a plurality of wireless communications to the wireless receiver 5603. This first wireless communication may include instructions to activate light source 5611. Receipt of the first user input by the computing device 5620 (e.g., by application 5622) may also cause the computing device 5620 (e.g., the application 5622) to load a new display page, such as by switching display 5624 from page 5010 of FIG. 5 to page 5110 of FIG. 6.

The first wireless communication may be received by wireless receiver 5603 and, in turn, by processing components 5630. In response to receipt of the first wireless communication, processing components 5630 may activate light source 5611. Light source 5611 may be activated by being changed to one or more active states, such as to start emitting light (e.g., by being powered-on), to start flashing, to change color (e.g., to change to a green color or another designated color associated with an active state), and the like. In the example of FIG. 6, light source 5611 may be activated by being caused to emit light, such as by being powered-on. Specifically, the light source 5611 may initially be unpowered, and the light source 5611 may remain unpowered until the first wireless communication is received by processing components 5630. When the first wireless communication is received by processing components 5630, the light source 5611 may be powered-on. Switch 5601 is an electrical switch that may control the flow of power from the battery 5604 to the light source 5611. The switch 5601 may be initially in an open state (which does not allow power to be provided to the light source 5611) and then, upon receipt of the first wireless communication by the processing components 5630, moved to a closed state (which does allow power to be provided to the light source 5611). In some examples, the processing components 5630 may, upon receipt of the first wireless communication, provide a signal to close the switch 5601 and allow power to flow to the light source 5611. Processing components 5630 may additionally or alternatively provide one or more other signals to cause light source 5611 to assume other active states, such as to flash, change color, etc.

In one specific example, light source 5611 may be light source 5021 of FIG. 6. The activation of light source 5611 may be a visual cue to perform a first step of a plurality of steps of instructions for use of medication delivery device 5610, such as to delivery medication to a user. In one specific example, the first step may be removing a cap of the medication delivery device 5610. As described above, activation of light source 5021 of FIG. 6 may be a visual cue to remove cap 320 from device 300.

Upon performing the first step (e.g., removing the cap), a user may provide second user input to application 5622 of computing device 5620. The second user input may indicate, to the computing device 5620, that the first step has been performed. For example, the second user input may include a selection of forward button 5012 of page 5110 of FIG. 6. Upon receipt of the second user input, the application 5622 may cause the wireless transmitter 5621 to transmit a second wireless communication of the plurality of wireless communications to the medication delivery device 5610. This second wireless communication may include instructions to activate light source 5612 (and optionally to deactivate light source 5611). Receipt of the second user input by the computing device 5620 (e.g., by application 5622) may also cause the computing device 5620 (e.g., the application 5622) to load a new display page, such as by switching display 5624 from page 5110 of FIG. 6 to page 5210 of FIG. 7.

The second wireless communication may be received by wireless receiver 5603 and, in turn, by processing components 5630. In response to receipt of the second wireless communication, processing components 5630 may deactivate light source 5611. Light source 5611 may be deactivated by being removed from a state that it assumed when it was activated. Thus, light source 5611 may be deactivated by being caused to stop emitting light (e.g., by being powered-off), to stop flashing, to change color (e.g., to change back to a color it had before it was activated), and the like. For example, in some cases, the processing components 5630 may, upon receipt of the second wireless communication, provide a signal to open the switch 5601 and prevent power from flowing to the light source 5611, thereby causing light source 5611 to be powered-off and to stop emitting light.

Additionally, in response to receipt of the second wireless communication, processing components 5630 may also activate light source 5612. Light source 5612 may be activated by being changed to one or more active states, such as to start emitting light (e.g., by being powered-on), to start flashing, to change color (e.g., to change to a green color or another designated color associated with an active state), and the like. In the example of FIG. 6, light source 5612 may be activated by being caused to emit light, such as by being powered-on. Specifically, the light source 5612 may initially be unpowered, and the light source 5612 may remain unpowered until the second wireless communication is received by processing components 5630. When the second wireless communication is received by processing components 5630, the light source 5612 may be powered-on. Switch 5602 is an electrical switch that may control the flow of power from the battery 5604 to the light source 5612. The switch 5602 may be initially in an open state (which does not allow power to be provided to the light source 5612) and then, upon receipt of the second wireless communication by the processing components 5630, moved to a closed state (which does allow power to be provided to the light source 5612). In some examples, the processing components 5630 may, upon receipt of the second wireless communication, provide a signal to close the switch 5602 and allow power to flow to the light source 5612. Processing components 5630 may additionally or alternatively provide one or more other signals to cause light source 5611 to assume other active states, such as to flash, change color, etc.

In one specific example, light source 5612 may be light source 5024 of FIG. 7. The activation of light source 5612 may be a visual cue to perform a second step of a plurality of steps of instructions for use of medication delivery device 5610. In one specific example, the second step may be an instruction to place the medication delivery device 5610 on the user's skin. As described above, activation of light source 5041 of FIG. 9 may be a visual cue to place device 300 on the user's skin.

As described above, although not shown in FIG. 11, the lighting circuit 5600 may include any number of additional light sources and respective switches. These additional light sources may also be activated based on additional communications received, by the wireless receiver 5603, from computing device 5620. Moreover, these additional communications may be transmitted based on user input to the computing device 5620, such as user input that indicates that the user has performed a previous step in a designated sequence of steps.

In some examples, any, or all, of the lighting circuit components (e.g., wireless receiver 5603, light source 5611, switch 5601, light source 5612, switch 5602, processing components 5630, battery 5604) of the lighting circuit 5600 (as well as other lighting circuits described herein) may be implemented, in whole or in part, using one or more integrated circuit (IC) components, such as one or more IC chips. The integrated circuit components may be connected to one or more circuit boards, such as a printed circuit board and/or flexible circuit board. The components may communicate and/or interact with one another via one or more electrical connections, for example via the one or more circuit boards. Any, or all, of the lighting circuit components of the lighting circuit 5600 shown in FIG. 11 (as well as other lighting circuits described herein) may optionally include one or more processing components (e.g., integrated with or separate from processing components 5630) and/or one or more memory components. The memory components can be volatile (such as some types of RAM), non-volatile (such as ROM, flash memory, etc.), or a combination thereof.

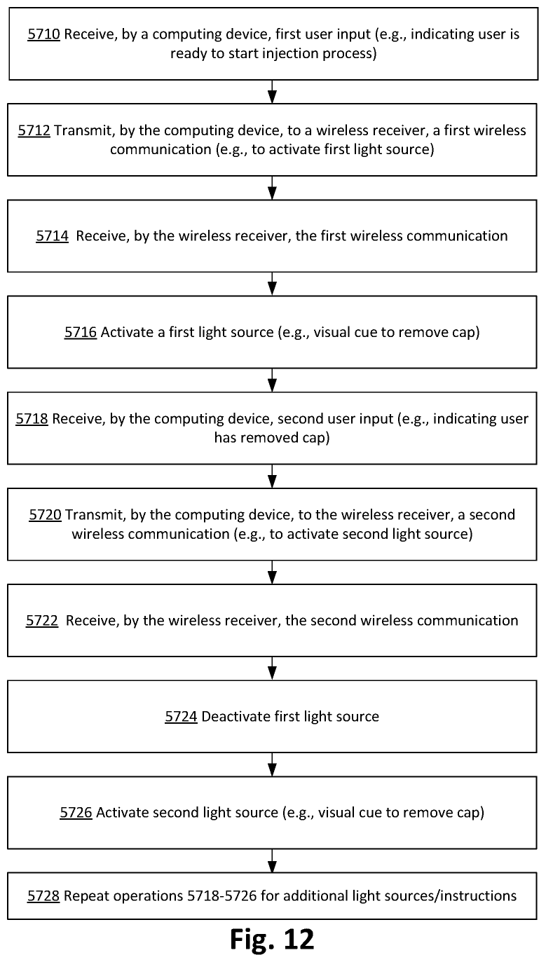

Referring now to FIG. 12, a process for light-based visual cueing of medication delivery instructions will now be described in detail. The process of FIG. 12 is initiated at operation 5710, at which first user input is received by a computing device. For example, as described above, the first user input may be received by application 5622 of computing device 5620. In one specific example, the first user input may indicate that the user is ready to start the injection process. For example, this may include a selection of forward button 5012 of page 5010 of FIG. 5.

At operation 5712, a first wireless communication is transmitted, by the computing device, to a wireless receiver that is integrated with a medication delivery device, such as a medication injection device. The first wireless communication may be transmitted based on the first user input to the computing device. For example, as described above, based on receipt of the first user input, the application 5622 may cause the wireless transmitter 5621 to transmit the first wireless communication to the wireless receiver 5603 that is integrated with medication delivery device 5610. The first wireless communication may include control instructions to activate the first light source. Receipt of the first user input by the computing device 5620 (e.g., by application 5622) may also cause the computing device 5620 (e.g., the application 5622) to load a new display page, such as by switching display 5624 from page 5010 of FIG. 5 to page 5110 of FIG. 6.

At operation 5714, the first wireless communication is received, by the wireless receiver, from the computing device. As described above, the first wireless communication is received via a wireless communication protocol, such as a short-range wireless communication protocol (e.g., Bluetooth, Near-Field Communication (NFC), and the like).

At operation 5716, a first light source is activated based on the first wireless communication. The first light source may be, for example, an OLED, OLED display, LED, or another type of light source. The first light source may be activated by being changed to one or more active states, such as to start emitting light (e.g., by being powered-on), to start flashing, to change color (e.g., to change to a green color or another designated color associated with an active state), and the like. As described above, in the example of FIG. 6, the first wireless communication may be received by wireless receiver 5603 and, in turn, by processing components 5630. In response to receipt of the first wireless communication, processing components 5630 may activate light source 5611. In the example of FIG. 6, light source 5611 may be activated by being caused to emit light, such as by being powered-on. Specifically, the light source 5611 may initially be unpowered, and the light source 5611 may remain unpowered until the first wireless communication is received by processing components 5630. When the first wireless communication is received by processing components 5630, the light source 5611 may be powered-on. Switch 5601 is an electrical switch that may control the flow of power from the battery 5604 to the light source 5611. The switch 5601 may be initially in an open state (which does not allow power to be provided to the light source 5611) and then, upon receipt of the first wireless communication by the processing components 5630, moved to a closed state (which does allow power to be provided to the light source 5611). In some examples, the processing components 5630 may, upon receipt of the first wireless communication, provide a signal to close the switch 5601 and allow power to flow to the light source 5611.

The activating of the first light source may be a first visual cue to perform a first step of a plurality of steps of instructions for use of a medication delivery device. The first step may be, for example, removing a cap, placing the medication delivery device on skin of the user, pushing down, keep pushing until a click is heard, or lifting the medication delivery device up. In one specific example, the first light source may be light source 5021 of FIG. 6. Also, in one specific example, the first step may be removing a cap of the medication delivery device 5610. As described above, activation of light source 5021 of FIG. 6 may be a visual cue to remove cap 320 from device 300.

Thus, the first light source may be activated in correspondence with (e.g., in synchronicity with) the first step being displayed by the computing device. The term synchronicity, as used herein, means that light source activation and the initiation of the display of the step by the computing device occur at the same time or nearly at the same time (e.g., with a minimal delay due to wireless transmission time, etc.). For example, as shown in FIG. 6, light source 5021 (which is a cue to remove the cap) may illuminate in correspondence with (e.g., in synchronicity with) loading of page 5110 including description 5101 (which instructs the user to remove the cap). As described above, receipt of the first user input may trigger application 5622 to cause the wireless transmitter 5621 to transmit the first wireless communication to the wireless receiver 5603 that is integrated with medication delivery device 5610. The first wireless communication may include control instructions to activate the first light source (e.g., light source 5021). Receipt of the first user input by the computing device 5620 (e.g., by application 5622) may also cause the computing device 5620 (e.g., the application 5622) to load a new display page, such as by switching display 5624 from page 5010 of FIG. 5 to page 5110 of FIG. 6. Additionally, light source 5021 may also be deactivated when the user presses the forward button 5012 of page 5110 of FIG. 6, which may also cause display 5624 to switch from page 5110 of FIG. 6 to page 5210 of FIG. 7.

At operation 5718, second user input is received by the computing device. For example, as described above, the second user input may be received by application 5622 of computing device 5620. For example, upon performing the first step (e.g., removing the cap), a user may provide second user input to application 5622 of computing device 5620. The second user input may indicate, to the computing device 5620, that the first step has been performed. For example, the second user input may include a selection of forward button 5012 of page 5110 of FIG. 6.

At operation 5720, a second wireless communication is transmitted, by the computing device, to the wireless receiver that is integrated with the medication delivery device. The second wireless communication may be transmitted based on the second user input to the computing device that indicates that the first step has been performed. For example, as described above, based on receipt of the second user input, the application 5622 may cause the wireless transmitter 5621 to transmit the second wireless communication to the wireless receiver 5603 that is integrated with the medication delivery device 5610. The second wireless communication may include control instructions to activate the second light source (and optionally to deactivate the first light source). Receipt of the second user input by the computing device 5620 (e.g., by application 5622) may also cause the computing device 5620 (e.g., the application 5622) to load a new display page, such as by switching display 5624 from page 5110 of FIG. 6 to page 5210 of FIG. 7.

At operation 5722, the second wireless communication is received, by the wireless receiver, from the computing device. As described above, the second wireless communication is received via the wireless communication protocol.

At operation 5724, the first light source is deactivated based on the second wireless communication. The first light source may be deactivated by being removed from a state that it assumed when it was activated. Thus, for example, the first light source may be deactivated by being caused to stop emitting light (e.g., by being powered-off), to stop flashing, to change color (e.g., to change back to a color it had before it was activated), and the like. As described above with respect to FIG. 6, the second wireless communication may be received by wireless receiver 5603 and, in turn, by processing components 5630. In response to receipt of the second wireless communication, the processing components 5630 may provide a signal to open the switch 5601, thereby powering-off the light source 5611 and causing the light source 5611 to stop emitting light.

At operation 5726, a second light source is activated based on the second wireless communication. The second light source may be, for example, an OLED, OLED display, LED, or another type of light source. The second light source may be activated by being changed to one or more active states, such as to start emitting light (e.g., by being powered-on), to start flashing, to change color (e.g., to change to a green color or another designated color associated with an active state), and the like. As described above, in the example of FIG. 6, in response to receipt of the second wireless communication, processing components 5630 may also activate light source 5612. In the example of FIG. 6, light source 5612 may be activated by being caused to emit light, such as by being powered-on. Specifically, the light source 5612 may initially be unpowered, and the light source 5612 may remain unpowered until the second wireless communication is received by processing components 5630. When the second wireless communication is received by processing components 5630, the light source 5612 may be powered-on. Switch 5602 is an electrical switch that may control the flow of power from the battery 5604 to the light source 5612. The switch 5602 may be initially in an open state (which does not allow power to be provided to the light source 5612) and then, upon receipt of the second wireless communication by the processing components 5630, moved to a closed state (which does allow power to be provided to the light source 5612). In some examples, the processing components 5630 may, upon receipt of the second wireless communication, provide a signal to close the switch 5602 and allow power to flow to the light source 5612.

The activating of the second light source may be a second visual cue to perform a second step of the plurality of steps of instructions for use of the medication delivery device. The second step may be, for example, removing a cap, placing the medication delivery device on skin of the user, pushing down, keep pushing until a click is heard, or lifting the medication delivery device up. In one specific example, second light source may be light source 5024 of FIG. 7. Also, in one specific example, the second step may be placing the medication delivery device on the user's skin. As described above, activation of light source 5024 of FIG. 7 may be a visual cue to place the medication delivery device on the user's skin.

Thus, the second light source may be activated in correspondence with (e.g., in synchronicity with) the second step being displayed by the computing device. For example, as shown in FIG. 7, light source 5024 (which is a cue to place the medication delivery device on the user's skin) may illuminate in correspondence with (e.g., in synchronicity with) loading of page 5210 including description 5201 (which instructs the user to cue to place the medication delivery device on the user's skin). As described above, receipt of the second user input may trigger application 5622 to cause the wireless transmitter 5621 to transmit the second wireless communication to the wireless receiver 5603 that is integrated with medication delivery device 5610. The second wireless communication may include control instructions to activate the second light source (e.g., light source 5024). Receipt of the second user input by the computing device 5620 (e.g., by application 5622) may also cause the computing device 5620 (e.g., the application 5622) to load a new display page, such as by switching display 5624 from page 5110 of FIG. 6 to page 5210 of FIG. 7. Additionally, light source 5024 may also be deactivated when the user presses the forward button 5012 of page 5110 of FIG. 6, which may also cause display 5624 to switch from page 5210 of FIG. 7 to page 5310 of FIG. 8.

As described above, the first step and the second step may be included in a plurality of steps of instructions for use of a medication delivery device. In some examples, the plurality of steps may be performed in a designated sequence. For example, a user may remove a cap, and then place the medication delivery device on skin of the user, and then push down on the handle of the medication delivery device, and then keep pushing until a click is heard, and then lift the medication delivery device up. In some examples, the second step may immediately follow the first step in the designated sequence. In one specific example, the first step may be removing a cap, and the second step may be placing the medication delivery device on skin of the user. In another specific example, the first step may be placing the medication delivery device on skin of the user, and the second step may be pushing down on the handle of the medication delivery device. In yet another specific example, the first step may be to keep pushing until a click is heard, and the second step may be lifting the medication delivery device up. In some examples, activating of the second light source may indicate that the injection is complete. For example, activating of light source 5023 of FIG. 10 may indicate that the injection is complete. Additionally, in some examples, the computing device may display an indication that the injection is complete. For example, description 5501 of FIG. 10 indicates that the injection is complete.

At operation 5728, the prior operations 5718-5726 may be repeated for each additional light source (and respective step of the instructions for use of the medication delivery device). For example, additional light sources may be activated based on additional communications received, by the wireless receiver, from the computing device. Moreover, these additional communications may be transmitted based on user input to the computing device, such as user input that indicates that the user has performed a previous step in a designated sequence of steps.

While example embodiments of devices for executing the disclosed techniques are described herein, the underlying concepts can be applied to any system capable of performing the techniques described herein. Thus, the methods and apparatuses described herein can be implemented, or certain aspects or portions thereof, can take the form of program code (e.g., instructions) embodied in tangible non-transitory storage media (e.g., the one or more memory components described above), including a processor-readable or machine-readable storage medium, wherein, when the program code (e.g., instructions) is loaded into and executed by a machine, the machine becomes an apparatus for performing the techniques described herein.

While the techniques described herein can be implemented and have been described in connection with the various embodiments of the various figures, it is to be understood that other similar embodiments can be used or modifications and additions can be made to the described embodiments without deviating therefrom. For example, it should be appreciated that the steps disclosed above can be performed in the order set forth above, or in any other order as desired. The techniques described herein should not be limited to any single embodiment, but rather should be construed in breadth and scope in accordance with the appended claims.

1. A light-based medication delivery cueing system, comprising:
a wireless receiver configured to receive a plurality of wireless communications from a computing device via a wireless communication protocol;
a first light source that is activated based on a first wireless communication of the plurality of wireless communications, wherein activating of the first light source is a first visual cue to perform a first step of a plurality of steps of instructions for use of a medication delivery device, and wherein the first light source is activated in correspondence with the first step being displayed by the computing device; and
a second light source that is activated based on a second wireless communication of the plurality of wireless communications, wherein activating of the second light source is a second visual cue to perform a second step of the plurality of steps associated with the instructions for use, wherein the second light source is activated in correspondence with the second step being displayed by the computing device, and wherein the second wireless communication is transmitted based on user input to the computing device.

2. The light-based medication delivery cueing system of claim 1, wherein the wireless receiver, the first light source, and the second light source are included in one or more adhesive labels that are attached to the medication delivery device.

3. The light-based medication delivery cueing system of claim 1, wherein the wireless receiver, the first light source, and the second light source are included within the medication delivery device.

4. The light-based medication delivery cueing system of claim 1, wherein the medication delivery device is a medication injection device.

5. The light-based medication delivery cueing system of claim 1, wherein the first light source is an organic light-emitting diode (OLED).

6. The light-based medication delivery cueing system of claim 1, wherein the wireless communication protocol is Bluetooth.

7. The light-based medication delivery cueing system of claim 1, wherein the wireless communication protocol is Near-Field Communication (NFC) protocol.

8. The light-based medication delivery cueing system of claim 1, wherein the computing device comprises a smartphone, a tablet, or a laptop.

9. The light-based medication delivery cueing system of claim 1, wherein the plurality of steps is performed in a designated sequence.

10. The light-based medication delivery cueing system of claim 9, wherein the second step immediately follows the first step in the designated sequence.

11. The light-based medication delivery cueing system of claim 1, wherein the first step comprises removing a cap, to placing the medication delivery device on skin of a user, pushing down, keep pushing until a click is heard, or lifting the medication delivery device up.

12. The light-based medication delivery cueing system of claim 1, wherein the second step comprises removing a cap, to placing the medication delivery device on skin of a user, pushing down, keep pushing until a click is heard, or lifting the medication delivery device up.

13. The light-based medication delivery cueing system of claim 1, wherein the user input indicates that the first step has been performed.

14. The light-based medication delivery cueing system of claim 1, wherein activating of the second light source indicates that an injection is complete.

15. The light-based medication delivery cueing system of claim 1, wherein the computing device displays an indication that an injection is complete.

16. The light-based medication delivery cueing system of claim 1, wherein the first light source is activated by at least one of causing the first light source to emit light, causing the first light source to flash, or causing the first light source to change color.

17. The light-based medication delivery cueing system of claim 1, wherein the second light source is activated by at least one of causing the second light source to emit light, causing the second light source to flash, or causing the second light source to change color.

18. A light-based medication delivery cueing method comprising:
receiving, by a wireless receiver, from a computing device, via a wireless communication protocol, a first wireless communication;
activating, based on the first wireless communication, a first light source, wherein activating of the first light source is a first visual cue to perform a first step of a plurality of steps of instructions for use of a medication delivery device, and wherein the first light source is activated in correspondence with the first step being displayed by the computing device;
receiving, by the wireless receiver, from the computing device, via the wireless communication protocol, a second wireless communication, wherein the second wireless communication is transmitted based on user input to the computing device; and
activating, based on the second wireless communication, a second light source, wherein activating of the second light source is a second visual cue to perform a second step of the plurality of steps associated with the instructions for use, and wherein the second light source is activated in correspondence with the second step being displayed by the computing device.

19-27. (canceled)

28. The light-based medication delivery cueing method of claim 18, wherein the first step comprises removing a cap, to placing the medication delivery device on skin of a user, pushing down, keep pushing until a click is heard, or lifting the medication delivery device up.

29. (canceled)

30. The light-based medication delivery cueing method of claim 18, wherein the user input indicates that the first step has been performed.

31-34. (canceled)
